(19) United States
(12) Patent Application Publication
(10) Pub. No.: US 2006/0014143 A1
(43) Pub. Date: Jan. 19, 2006

(54) Preparation of potent macrophage activating factors derived from cloned vitamin D binding protein and its domain and their therapeutic usage for cancer, HIV-infection and osteopetrosis

(76) Inventor: Nobuto Yamamoto, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/201,560

(22) Filed: Sep. 21, 2005

Related U.S. Application Data
(63) Continuation-in-part of application No. 09/826,463, filed on Apr. 5, 2001.
Application No. 09/826,463 is a continuation of application No. 08/618,485, filed on Mar. 19, 1996, now Pat. No. 6,410,269.
Application No. 08/618,485 is a continuation-in-part of application No. 08/478,121, filed on Jun. 7, 1995, now Pat. No. 5,620,846.

(51) Int. Cl. C12Q 1/70; C12P 21/04; C12N 5/06; C12N 15/86

(52) U.S. Cl. 435/5; 435/69.6; 435/320.1; 435/456; 435/348

(57) ABSTRACT

Vitamin D-binding protein (Gc protein) and its small domain (approximately ⅕ of the Gc peptide also known as domain III) were cloned via a baculovirus vector. The cloned Gc protein and the cloned domain (Cd) peptide were treated with immobilized β-galactosidase and sialidase to yield macrophage activating factors, GcMAFc and CdMAF, respectively. These cloned macrophage activating factors and GcMAF are to be used for therapy of cancer, HIV-infection and osteopetrosis, and may also be used as adjuvants for immunization and vaccination.

### CROSS-REFERENCE TO RELATED APPLICATIONS

This application is a continuation-in-part application of application Ser. No. 09/826,463 filed Apr. 5, 2001 entitled PREPARATION OF POTENT MACROPHAGE ACTIVATING FACTORS DERIVED FROM CLONED VITAMIN D BINDING PROTEIN AND ITS DOMAIN AND THEIR THERAPEUTIC USAGE FOR CANCER, HIV-INFECTION AND OSTEOPETROSIS, which is a continuation of application Ser. No. 08/618,485 filed Mar. 19, 1996 entitled PREPARATION OF POTENT MACROPHAGE ACTIVATING FACTORS DERIVED FROM CLONED VITAMIN D BINDING PROTEIN AND ITS DOMAIN AND THEIR THERAPEUTIC USAGE FOR CANCER, HIV-INFECTION AND OSTEOPETROSIS which is a continuation-in-part of application Ser. No. 08/478,121 filed Jun. 7, 1995 entitled, DIAGNOSTIC AND PROGNOSTIC INDICES FOR CANCER AND AIDS, the entire disclosure of which is incorporated by reference herein.

### FIELD OF INVENTION

This invention relates to potent macrophage activating factors, prepared by oligosaccharide digestion of the cloned vitamin D binding protein (Gc protein) and the cloned Gc protein domain II, and the use of these macrophage activating factors for various cancer, HIV-infection and osteopetrosis, and as adjuvants for immunization and vaccination.

| | |
|---|---|
| Gc | Vitamin D₃-binding protein |
| MAF | macrophage activating factor |
| GcMAF | Gc protein-derived macrophage activating protein |
| GcMAFc | cloned Gc protein-derived macrophage activating factor |
| Gc domain III | domain III region of Gc protein |
| CdMAF | cloned domain III-derived macrophage activating factor |

### BRIEF SUMMARY OF THE INVENTION

Vitamin D-binding protein (Gc protein) and its small domain (approximately ⅕ of the Gc peptide also known as domain III) were cloned via a baculovirus vector. The cloned Gc protein and the cloned domain (Cd) peptide were treated with immobilized β-galactosidase and sialidase to yield macrophage activating factors, GcMAFc and CdMAF, respectively. These cloned macrophage activating factors and GcMAF are to be used for therapy of cancer, HIV-infection and osteopetrosis, and may also be used as adjuvants for immunization and vaccination.

### BRIEF DESCRIPTION OF THE DRAWINGS

The invention will be described in conjunction with the following drawings in which like reference numerals designate like elements and wherein:

FIG. 1a is a schematic illustration of the formation of macrophage activating factor (MAF).

FIG. 1b is a schematic illustration of the deglycosylation of Gc protein in a cancer or HIV-infected patient's blood stream.

FIG. 2 shows the correlation between plasma α-N-acetylgalactosaminidase activity and tumor burden (total cell counts) in the peritoneal cavity of Ehrlich ascites tumor.

FIG. 3 shows the amino acid sequence of cloned GcMAF which is SEQ ID NO:1 which is the entire cloned Gc protein.

FIG. 4 shows the construction of the DNA fragment encoding the leader sequence of EcoRI fragment E1 and domain III regions of the Gc protein; A, the entire cDNA for Gc protein; B, the construct to be inserted into the non-fusion vector; the shaded area indicates the compressed regions of about 1,000 base pairs (bp).

FIG. 5 shows the 89 amino acid sequence, SEQ ID NO:2, of the cloned domain III (CdMAF₁), using the non-fusion vector.

FIG. 6 shows the baculovirus fusion vector for cloning the domain III of Gc protein.

FIG. 7 shows the 94 amino acid sequence, SEQ ID NO:3, of the cloned domain III (CdMAF₂), using the fusion vector.

FIG. 8A shows the therapeutic effect of GcMAF in accordance with the present invention on adult persons suffering from prostate cancer.

FIG. 8B shows the therapeutic effect of GcMAF in accordance with the present invention on adult persons suffering from breast cancer.

FIG. 8C shows the therapeutic effect of GcMAF in accordance with the present invention on adult persons suffering from colon cancer.

FIG. 8D shows the therapeutic effect of GcMAF in accordance with the present invention on adult persons suffering from leukemia.

### DETAILED DESCRIPTION OF THE INVENTION

A. Inflammatory Response Results in Activation of Macrophages

Inflammation results in the activation of macrophages. Inflamed lesions release lysophospholipids. The administration into mice of doses (5-20 μg/mouse) of lysophosphatidycholine (lyso-Pc) and other lysophospholipids induced a greatly enhanced phagocytic and superoxide generating capacity of macrophages (Ngwenya and Yamamoto, Proc. Soc. Exp. Biol. Med. 193:118, 1990; Yamamoto et al., Inf. 1 mm. 61:5388, 1993; Yamamoto et al., Inflammation. 18:311, 1994).

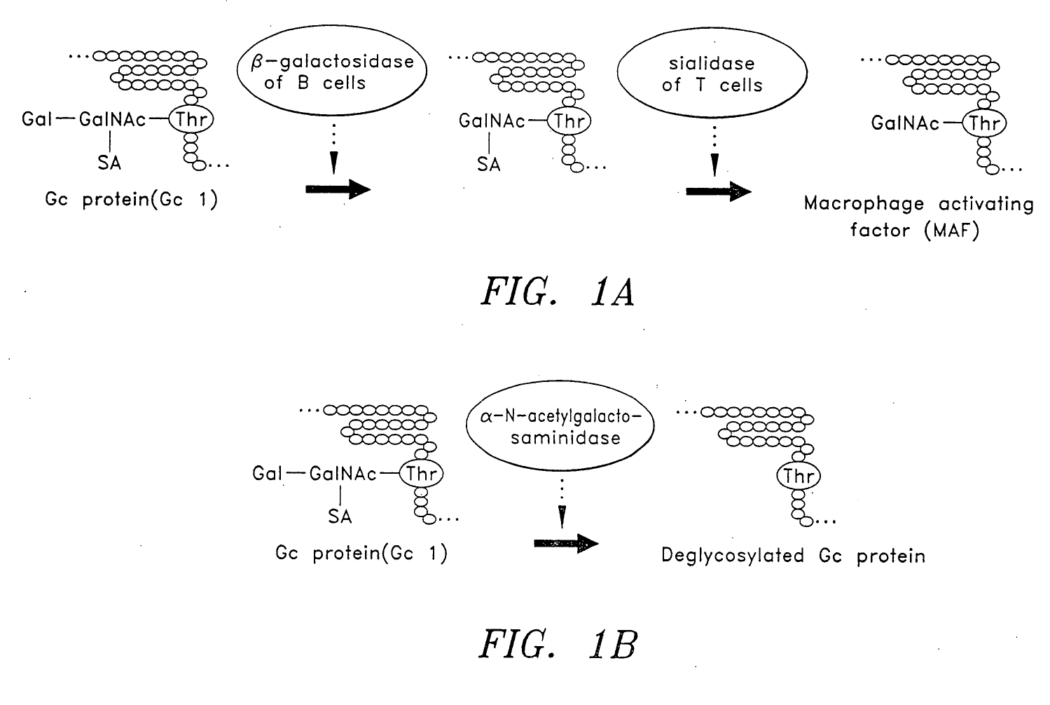

This macrophage activation requires participation of B and T lymphocytes and serum vitamin D binding protein (DBP; human DBP is known as Gc protein). In vitro activation of mouse peritoneal macrophages by lyso-Pc requires the step-wise modification of Gc protein by β-galactosidase of lyso-Pc-treated B cells and sialidase of T cells to generate the macrophage activating factor (MAF), a protein with N-acetylgalactosamine as the remaining sugar moiety (FIG. 1a) (Yamamoto et al., Pro. Natl. Acad. Sci. USA. 88:8539, 1991; Yamamoto et al., J. Immunol. 151:2794, 1933; Naraparaju and Yamamoto, Immunol. Lett. 43:143, 1994). Thus, Gc protein is a precursor for MAF.

Incubation of Gc protein with immobilized β-galactosidase and sialidase generates a remarkably high tittered MAF (GcMAF) (Yamamoto et al. Proc. Natal. Acad. SCI. USA. 88:8539, 1991; Yamamoto et al., J. Immunol. 151:2794, 1993; Naraparaju and Yamamoto, Immunol Lett., 43:143, 1994; U.S. Pat. No. 5,177,022). Administration of a minute amount (10 pg/mouse; 100 ng/human) of GcMAF resulted in greatly enhanced phagocytic and superoxide generating capacities of macrophages.

When peripheral blood monocytes/macrophages (designated as macrophages hereafter) of 258 cancer patients bearing various types of cancer were treated in vitro with 100 pg GcMAF/ml, macrophages of all cancer patients were activated for phagocytic and superoxide generating capacity. This observation indicates that cancer patient macrophages are capable of being activated. However, the MAF precursor activity of plasma Gc protein was lost or reduced in approximately 70% of this cancer patient population. Loss of the MAF precursor activity prevents generation of MAF. Therefore, macrophage activation cannot develop in certain cancer patients. Since macrophage activation is the first step in the immune development cascade, such cancer patients become immunosuppressed. This may explain at least in part why cancer patients die from overwhelming infection. Lost or reduced precursor activity of Gc protein was found to be due to deglycosylation of plasma Gc protein by α-N-acetylgalactosaminidase detected in cancer patient blood stream. Deglycosylated Gc protein cannot be converted to MAF (FIG. 1b)(Yamamoto et al., Cancer Res. 56:2827, 1976).

Similarly, when peripheral blood macrophages of 160 HIV-infected/AID patients were treated in vitro with 100 pg GcMAF/ml, macrophage of all patients were activated for phagocytic and superoxide generating capacity. However, the MAF precursor activity ofplasma Gc protein was low in approximately 35% of the HIV-infected patient population. As in cancer patients, these patients' plasma Gc protein is deglycosylated by α-N-acetylgalactosaminidase detected in HIV-infected patients (Yamamoto et al., AIDS Res. Human Ret., 11:1373, 1995).

Both cancer and HIV-infected patients having severely decreased precursor activity of plasma Gc protein carried large amounts of α-N-acetylgalactosaminidase while patients having moderately decreased precursor activity had moderate levels of α-N-acetylgalactosaminidase activities. Patients with high precursor activity, including asymptomatic HIV-infected patients, had low but significant levels of plasma α-N-acetylgalactosaminidase activity. Since a large amount (260 μg/ml) of Gc protein exists in the blood stream, a low level of the enzyme does not affect the precursor activity. Nevertheless, α-N-acetylgalactosaminidase activity was found in plasmas of all cancer and HIV-infected patients and had an inverse correlation with the precursor activity of their plasma Gc protein (Yamamoto et al., AIDS Res. Human Ret. 11:1373, 1995). Thus, increase in patient plasma α-N-acetylgalactosaminidase activity is responsible for decrease in the precursor activity of plasma Gc protein. These observations lead us to propose that plasma α-N-acetylgalactosaminidase plays a role in immunosuppression in cancer and HIV-infected/AID patients.

B. The Origin of Immunnosuppression

The source of the plasma α-N-acetylgalactosaminidase in cancer patients appeared to be cancerous cells. High α-N-acetylgalactosaminidase activities were detected in tumor tissue homogenates of various organs, including eleven different tumor tissues including 4 lung tumors, 3 breast tumors, 3 colon tumors and 1 cervical tumor, though the α-N-acetylgalactosaminidase activity varied from 15.9 to 50.8 mmoles/mg/min. Surgical removal of malignant lesions in human cancer results in a sudden decrease of plasma α-N-acetylgalactosaminidase activity with concomitant increase in the precursor activity, particularly if malignant cells are localized.) (Yamamoto et al., Cancer Res. 57:295, 1997).

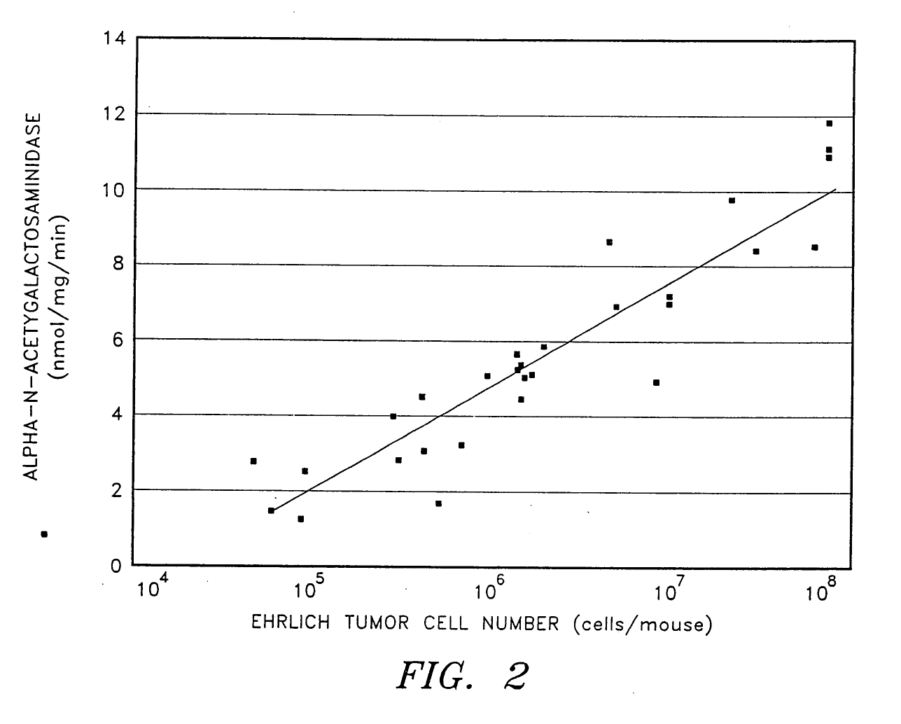

In a preclinical mouse tumor model, BALB/c mice were transplanted with 5×10⁵ Ehrlich ascites tumor cells/mice into peritoneal cavity and analyzed for serum α-N-acetylgalactosaminidase activity. When plasma enzyme level were measured as transplanted Ehrlich ascites tumor grew in mouse peritoneal cavity, the enzyme activity was directly proportional to tumor burden as shown in FIG. 2 (Koga et al., Pro. Soc. Exp. Biol. Med. 220:20, 1999). This was also confirmed with a nude mouse transplanted with KB cells (human oral squamous cell carcinoma cell line) (Yamamoto et al., Cancer Res. 57:295, 1997). Serum α-N-acetylgalactosaminidase activity increased as tumor size (measured by weight) of the solid tumor increased. Thus, plasma α-N-acetylgalactosaminidase activity may be used as a prognostic index to monitor the progress of therapy.

Radiation therapy of human cancer decreased plasma α-N-acetylgalactosaminidase activity with a concomitant increase of precursor activity. This implies that radiation therapy decreases the number of cancerous cells capable of secreting α-N-acetylgalactosaminidase. These results also confirmed that plasma α-N-acetylgalactosaminidase activity has an inverse correlation with the MAF precursor activity of Gc protein. Even after surgical removal of tumor lesions in cancer patients, most post-operative patients carried significant amounts of α-N-acetylgalactosaminidase activity in their blood stream. The remnant cancerous lesions in these post-operative patients cannot be detectable by any other procedures, e.g., X-ray, scintigraphy, etc. This most sensitive enzyme assay has been used by the instant inventor as prognostic index during the course of GcMAF therapy for treating cancer.

HIV-infected cells appeared to secrete α-N-acetylgalactosaminidase. When peripheral blood mononuclear cells (PBMC) of HIV-infected patients were cultured and treated with mitomycin as a provirus inducing agent (Sato et al., Arch. Virol. 54:33, 1977), α-N-acetylgalactosaminidase was secreted into culture media. These results led us to suggest that α-N-acetylgalactosaminidase is a virus coded product. In fact, HIV-envelope protein gp120 appears to carry the α-N-acetylgalactosaminidase activity.

C. A Defect in Macrophage Activation Cascade Manifests Osteopetrosis

An inflammation-primed macrophage activation cascade has been defined as a major process leading to the production of macrophage activating factor. Activation of other phagocytes such as osteoclasts shares the macrophage activation cascade (Yamamoto et al., J. Immunol. 152:5100, 1994). Thus, a defect in the macrophage activation cascade results in lack of activation in osteoclasts.

Autosomal recessive osteopetrosis is characterized by an excess accumulation of bone throughout the skeleton as a result of dysfunctional osteoclasts, resulting in reduced bone resorption (Marks, Clin. Orthop. 189:239, 1984). In animal models of osteopetrosis, depending on the degree of osteoclast dysfunction, marrow cavity development and tooth eruption are either delayed or more commonly absent (Marks, Am. J. Med. Genet. 34:43, 1989). In human infantile osteopetrosis, death occurs within the first decade of life usually from overwhelming infection (Reeves, Pediatrics. 64:202, 1979), indicating immunosuppression. Accumulated evidence suggests that deficient or dysfunctional osteoclasts in osteopetrotic animals are often accompanied by deficiencies or dysfunctions of macrophages. The studies of the instant inventor on the activation of both osteoclasts and macrophages in the osteopetrotic mutations revealed that osteoclasts and macrophages can be activated by a common signaling factor, the macrophage activating factor and that a defect in β-galactosidase of B cells incapacitates the generation process of macrophage activating factor (Yamamoto et al., J. Immunol. 152:5100, 1994). Since GcMAF and its cloned derivatives bypass the function of lymphocytes and Gc protein and act directly on macrophages and osteoclasts, administration of these factors into osteopetrotic hosts should rectify the bone disorder. In fact the instant inventor has recently found that four administrations of purified cloned human macrophage activating factor (GcMAFc) (100 pg/week) to the op mutant mice beginning at birth for four weeks resulted in the activation of both macrophages and osteoclasts and subsequent resorption of the excess skeletal matrix.

D. Therapeutic Application of GcMAF and its Cloned Derivatives on Cancer

Despite defects in the macrophage activation cascade in cancer, HIV-infected and osteopetrotic patients, GcMAF bypasses the functions of lymphocytes and Gc protein and acts directly on macrophages (or osteoclasts) for activation. Macrophages have a potential to eliminate cancerous cells and HIV-infected cells when activated. When cancer patients were treated with 100 ng GcMAF/patient weekly for several months, GcMAF showed remarkable curative effects on a variety of human cancer indiscriminately.

Instead of obtaining of GcMAF from human blood source, it can be obtained from the cloned Gc protein or its small domain responsible for macrophage activation. The cloning Gc protein require a eukaryotic vector/host capable of the glycosylation of the cloned products. The Gc protein having a molecular weight of 52,000 and 458 amino acid residues is a multi-functional protein and carries three distinct domains (Cooke and Haddad, Endocrine Rev., 10:294), 1989.

Domain I interacts with vitamin D while domain III interacts with actin (Haddad et al., Biochem., 31:7174, 1992). Chemically and proteolytically fragmented Gc protein enabled the instant inventor to indicate that the smallest domain, domain III, contains an essential peptide for macrophage activation. Accordingly, both Gc protein and the entire domain III peptide were cloned by the use of a baculovirus vector and an insect host, and then treated with the immobilized β-galactosidase and sialidase to yield potent macrophage activating factors, designated GcMAFc and CdMAF, respectively. Like GcMAF, these cloned GcMAFc and CdMAF appear to have curative effects on cancer.

E. Requirement of Native Wild Type Gc Peptide Sequence for Preparation of Cloned Derivatives

In a preferred embodiment, the peptide sequence of GcMAF is the peptide sequence of native plasma Gc protein (SEQ ID NO.: 1), as opposed to a mutant form of GcMAF. Thus, the peptide of GcMAFc should be the same peptide of native plasma Gc protein before treatment with immobilized β-galactosidase and sialidase. Protein synthesis in the cloning apparatus occasionally yields mutant Gc peptides having an amino acid substitution due to mistakes made during gene copying processes or during translation (possible translational infidelity). However, most of these mutant Gc peptides become functional GcMAFc obtained after treatment of these mutant peptides with immobilized β-galactosidase and sialidase and, therefore, are likely to be immunogenic thereby causing anaphylaxis in humans. Thus, only the cloned Gc protein having the wild type peptide sequence is to be used to generate the cloned GcMAFc of the instant invention. In order to determine if the cloned protein is wild-type Gc protein, the protein must be sequenced. In one embodiment, the protein is sequenced by mass spectroscopy or N-terminal sequencing. After sequencing the cloned Gc protein, one would be able to compare the sequenced protein or peptide to the wild-type amino acid sequence that is disclosed in SEQ ID NO.:1.

F. A Potent Adjuvant Activity of GcMAF for Immunization with Antigens or Vaccines

Macrophages are antigen presenting cells. Macrophages activated by GcMAF rapidly phagocytize target antigens or cells and present the processed antigens to antibody producing cells. A rapid development of a large amount of antibody secreting cells was observed immediately (1 to 4 days) after inoculation of small amount of GcMAF (100 pg/mouse) and sheep erythrocytes (SRBC). This finding indicates that and its cloned derivatives, GcMAFc and CdMAF, should serve as potent adjuvants for immunization and vaccination.

### Description of the Methods for Gene Cloning for Macrophage Activating Factors

A. Cloning of the cDNA of Gc Protein into an Insect Virus

A full length cDNA encoding the human Gc protein was isolated from a human liver cDNA library in bateriophage λgt11 (Clontech, Palo Alto, Calif.) by the use of pico Blue™ immunoscreening kit available from Stratagene of La Jolla, Calif. The baculoviral expression system in the insect cells takes advantages of several facts about the polyhedron protein: (a) it is expressed to very high levels in infected cells where it constitutes more than half of the total cellular protein late in the infection cycle; (b) it is nonessential for infection or replication of the virus, meaning that the recombinant virus does not require any helper function; (c) viruses lacking the polyhedron gene have distinct plaque morphology from viruses containing the cloned gene; and (d) unlike bacterial cells, the insect cell efficiently glycosylate the cloned gene products.

One of the advantages of this expression system is a visual screen allowing recombinant viruses to be distinguished and quantified. The polyhedron protein is produced at very high levels in the nuclei of infected cells late in the viral infection cycle. Accumulated polyhedron protein forms occlusion bodies that also contain embedded virus particles. These occlusion bodies, up to 15 μm in size, are highly refractile, giving them a bright shiny appearance that is readily visualized under a light microscope. Cells infected with recombinant viruses lack occlusion bodies. To distinguish recombinant virus from wild-type virus, the transfection supernatant (recombinant containing virus lysate) is plaqued onto a monolayer of insect cells. Plaques are then screened under a light microscope for the presence (indicate of wild-type virus) or absence (indicative of recombinant virus) of occlusion bodies.

Unlike bacterial expression systems, the baculovirus-based system is a eukaryotic expression system and thus uses many of the protein modification, processing such as glycosylation, and transport reactions present in higher eukaryotic cells. In addition, the baculoviral expression system uses a helper-independent virus that can be propagated to high titers in insect cells adapted for growth in suspension cultures, making it possible to obtain a large amount of recombinant protein with relative ease. The majority of the overproduced protein from insect cells remains soluble by contrast with the insoluble proteins often obtained from bacteria. Furthermore, the viral genome is large (130 kbp) and thus can accommodate large segments of foreign DNA. Finally, baculoviruses are noninfectious to vertebrates, their promoters have been shown to be inactive in mammalian cells (Carbonell et al., J. Virol., 56:153, 1985), which gives them a possible advantage over other systems when expressing oncogenes or potentially toxic proteins.

1) Choice of Baculoviral Vector

All available baculoviral vectors are pUC-based and confer ampicillin resistance. Each contains the polyhedron gene promoter, variable lengths of polyhedron coding sequence, and insertion site(s) for cloning the foreign gene of interest flanked by viral sequence that lie 5′ to the promoter and 3′ to the foreign gene insert. These flanking sequences facilitate homologous recombination between the vector and wild-type baculoviral DNA (Ausubel et al., Current Protocols in Mol. Biol. 1990). The major consideration when choosing the appropriate baculoviral expression vector is whether to express the recombinant as a fusion or non-fusion protein. Since glycosylation of Gc peptide requires a leader signal sequence for transfer of the peptide into the endoplasmic reticulum, the cDNA containing initiation codon (−16 Met) through the leader sequence to the +1 amino acid (leu) of the native Gc protein should be introduced to non-fusion vector with a polylinker carrying the EcoR1 site, pVL1393 (Invitrogen, San Diego, Calif.).

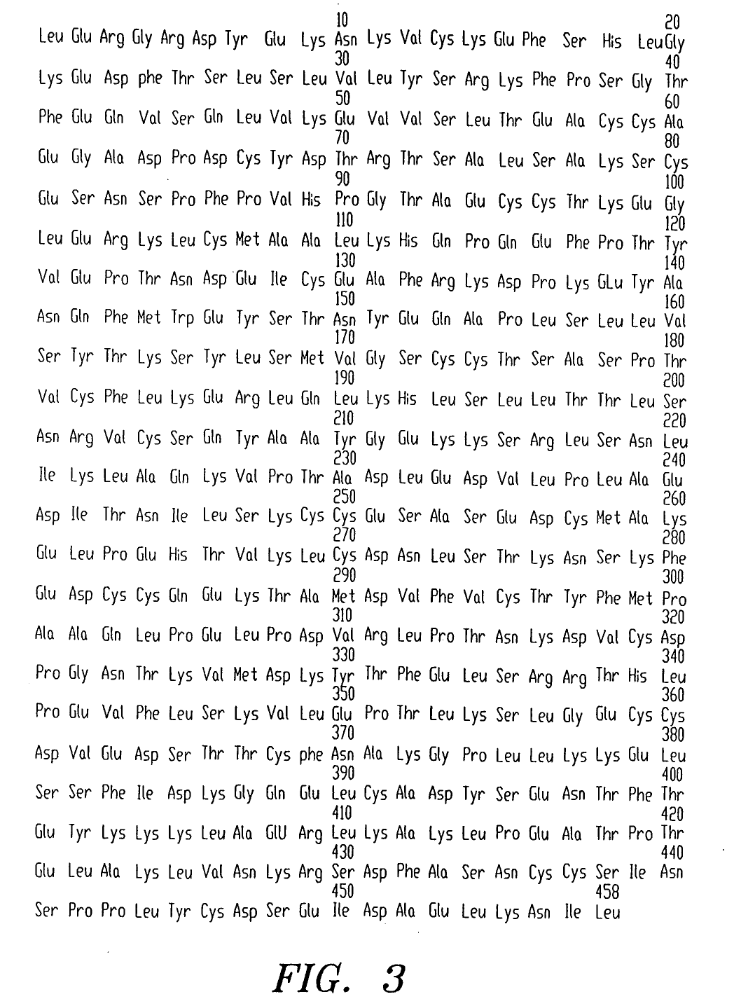

During partial digestion of the cDNA for Gc protein in λgt11 with EcoR1 enzyme, a full length Gc cDNA with EcoR1 termini was isolated electrophoretically, mixed with EcoR1-cut pVL1393, and ligated with T4 ligase. This construct in correct orientation should express the entire Gc peptide, a total of 458 amino acids (FIG. 3). To obtain the correct construction, competent E. coli, HB101 cells were transformed with pVL vector and selected for transformants on Luria broth agar plates containing ampicillin (LB/ampicillin plates). The DNA was prepared for the sequencing procedure to determine which colony contains the insert or gene with proper reading orientation, by first searching for the 3′ poly A stretch. The clones with 3′ poly A (from the poly A tail of mRNA) were then sequenced from the 5′ end to confirm the correct orientation of the full length DNA for the Gc peptide.

2) Co-Transfection of Insect Cells with the Cloned Plasmid DNA and Wild-Type Viral DNA

A monolayer (2.5×10⁶ cells in each of 25-cm² flasks) of Spodoptera frugiperda (Sf9) cells was co-transfected with a cloned plasmid (vector) DNA (2 μg) and a wild-type (Ac-MNPV) baculoviral DNA (10 μg) in 950 μl transfection buffer (Ausubel et al., In Curr Protocols in Mol. Biol. 1990). When the cells were cultured for 4 or 5 days, the transfection supernatant contained recombinant viruses.

3) Identification of Recombinant Baculovirus

The co-transfection lysates were diluted 10⁴, 10⁵ or 10⁶ and plated on Sf9 cells for cultivation for 4 to 6 days. After the plaques were well formed, plaques containing occlusion-negative cells were identified at a frequency of 1.3%. Several putative recombinant viral plaques were isolated and twice re-plaqued for purification. Pure recombinant viral plaque clones were isolated.

B. Analysis of Protein of Interest from Recombinant Baculovirus

1) Preparation of Recombinant Virus Lysate

An insect cell Sf9 monolayer (2.5×10⁶ cells per 25-cm² flask) was infected with a recombinant virus clone and cultured in 5 ml GIBCO serum-free medium (from GIBCO Biochemicals, Rockville, Md.) or medium supplemented with 0.1% egg albumin to avoid contamination of serum bovine vitamin D binding protein. The culture flasks were incubated at 27° C. and monitored daily for signs of infection. After 4 to 5 days, the cells were harvested by gently dislodging them from the flask and the cells and culture medium were transferred to centrifuge tubes and centrifuged for 10 min at 1000×g, 4° C. To maximize infection for recombinant protein production, Sf9 cells were grown in a 100-ml spinner suspension culture flask with 50 ml complete medium up to about 2×10⁶ cells/ml. The cells were harvested, centrifuged at 1000×g for 10 min and re-suspended in 10 to 20 ml serum-free medium containing recombinant virus at a multiplicity of infection (MOI) of 10. After 1 hour of incubation at room temperature, the infected cells were transferred to a 200-ml spinner flask containing 100 ml serum-free medium and incubated for 40 hr. More than 40% of the protein secreted was the protein of interest. The protein in the supernatant was isolated. 2) Qualitative Estimation of the Protein of Interest

Coomassie Blue staining of the SDS-polyacrylamide gel, loading 20 to 40 μg total cell protein per lane, was to estimate quantity of expressed protein. Because the samples contain cellular proteins, the recombinant protein was readily detected by comparison with uninfected cellular proteins.

3) Sequencing of Cloned GcMAFc Protein/Peptide

There are several protocols that may be used for protein sequencing which include but are not limited to the protocol are listed below.

Common Strategy For Protein Sequencing (MIT Biology Department, http://web.mit.edu/esgbio/www/7001 main.html)

Protein chemists follow a basic strategy when they attempt to determine the sequence of most proteins. The steps include:

a) Determine the Amino Acid Composition

In order to know which amino acids and how many of each amino acid there are in a polypeptide the peptide bonds must be broken. This can be accomplished with strong acids (i.e. 6N HCl) or strong bases or by exhaustive enzymatic digestion. By performing an acid hydrolysis or base hydrolysis experiment a minimum length for the polypeptide is obtained.

B) Break all Disulfide Bonds

Disulfide cross-links complicate the determination of amino acid sequences and usually are cleaved by reduction or oxidation before sequence analysis.

c) Perform an Initial N-Terminal Sequence Determination

In this reaction phenylisothiocyanate (PITC) reacts with the amino acid residue at the amino terminus under basic conditions (provided by n-methylpiperidine/methanol/water) to form a phenylthiocarbamyl derivative (PTC-protein). Trifluoroacetic acid then cleaves off the first amino acid as its anilinothialinone derivative (ATZ-amino acid) and leaves the new amino terminus for the next degradation cycle. The ATZ amino acid is then removed by extraction with N-butyl chloride and converted to a phenylthiohydantoin derivative (PTH-amino acid) with 25% TFA/water. Several by-products are also formed during the above Edman degradation chemistry. The PTH-amino acid is transferred to a reverse-phase C-18 column for detection at 270 nm. A standard mixture of 19 PTH-amino acids is also injected onto the column for separation (usually as the first cycle of the sequencing run). This chromatogram provides standard retention times of the amino acids for comparison with each Edman degradation cycle chromatogram. The HPLC chromatograms are collected using a computer data analysis system. To determine the amino acid present at a particular residue number, the chromatogram from the residues of interest is compared with the chromatogram from the previous residue by overlaying one on top of the other. From this, the amino acid for the particular residue can be determined. This process is repeated sequentially to provide the N-terminal sequence of the protein/peptide.

d) Fragmentation of the Peptide

Break the polypeptide into fragments by cleaving at specific amino acids. Several cleavage methods are available, each of which have different specificity (i.e. cleave at different amino acids).

e) Repeat Steps c and d to Determine Sub-Sequences and Create “Overlappings”

The initial cleavage is generally made as specific as possible in order to generate large peptide fragments. It is easy to arrange fewer fragments. These fragments can be positioned relative to one another after treatment of the original polypeptide by a second cleavage procedure that generates fragments whose sequences extend across the initial cleavage points (referred to as overlapping peptides). The amino acid sequence of each overlap peptide orders two or more of the original fragments.

f) Reconstruct the Original Protein

From the overlapping peptides and information gained from the original protein, it is possible to construct a unique sequence for the protein or polypeptide of interest. Your overlaps should be at least two amino acids in length.

g) Locate the Disulfide Bonds

No primary structure analysis of a cysteine-containing protein can be regarded as complete before the presence and location of disulfide bonds has been established.

4) Protein Sequencing Procedure Used for Sequencing Gc1 Peptide

a) Fragmentation of the Cloned Gc1 Peptide

Before peptide sequencing, the cloned Gc1 peptide was treated with cyanogen bromide (methionine specific) to yield 8 peptide fragments. The last (C-terminal) largest fragment carrying 131 amino acids was further fragmented by treating with thrombin (arginine specific protease) to yield four peptide fragments.

b) Sequencing Fragmented Cloned Peptide

Fragmented cloned Gc1 peptides were sequenced by the Edman degradation procedure.

The first step in the Edman degradation procedure is treatment of a polypeptide chain with phenylisothiocyanate. At pH 9.0, phenylisothiocyanate reacts with the N-terminus to produce phenylthiocarbamyl (PTC) derivative of the peptide. When PTC derivative is treated with anyhdrous trifluoroacetic acid (TFA), the peptide bond of the N-terminal residue is cleaved and a thiazolinone derivative of this residue is released and extracted without destruction of the other peptide bonds in the polypeptide chain. After treatment with aqueous acid, this extracted thiazolinone derivative is rearranged to form a stable phenylthiohydantoin (PTH) amino acid. Finally, this PTH derivative of N-terminal amino acid is identified by chromatography. This entire procedure was automatically repeated using amino acid (protein) sequencer to sequence the peptide.

5) Enzymatic Conversion of the Cloned Gc Protein to Macrophage Activating Factor (GcMAFc).

The cloned Gc protein (2 μg) with a molecular weight of 52,000 and 458 amino acid residues (FIG. 3) was isolated by electroeluter and treated with immobilized β-galactosidase and sialidase. The resultant cloned macrophage activating actor (GcMAFc) was added to mouse and human macrophages and assayed for phagocytic and superoxide generating capacity. Incubation of macrophages with 10 pg GcMAFc/ml for 3 hours resulted in a 5-fold increased phagocytic and a 15-fold increase in the superoxide generating capacity of macrophages.

C. I of a Domain Required for Macrophage Activation

I. Cloning procedure I: non-fusion vector.

1) Cloning the Domain Responsible for Macrophage Activation (CdMAF)

The entire cDNA sequence for Gc protein in λgt11, including 76 bp of the upstream 5′ flanking region and 204 bp of the 3′ flanking stretch, was fragmented by EcoR1 to yield four restriction fragments designated E1, 120; E2, 314; E3,482; and E4, 758 bp, respectively. Each was cloned into the EcoR1 site of the plasmid pSP65 from Promega (Madison, Wis.) by the method of Cooke and David (J. Clin. Invest., 76 2420, 1985). Although it was found that a region less than one half of the domain III was responsible for macrophage activation, small segments less than 40 amino acid residues cannot be expressed in the insect cells. Moreover, short peptides are rapidly degraded by proteases in human plasma and thus are not clinically useful. Accordingly, the entire domain III (approximately 80 amino acid residues) should be subcloned into an insect virus where I anticipate the efficient production and glycosylation of the peptide in the infected cells.

2) Subcloning cDNA Fragment into the Polyhedron Gene of Baculovirus

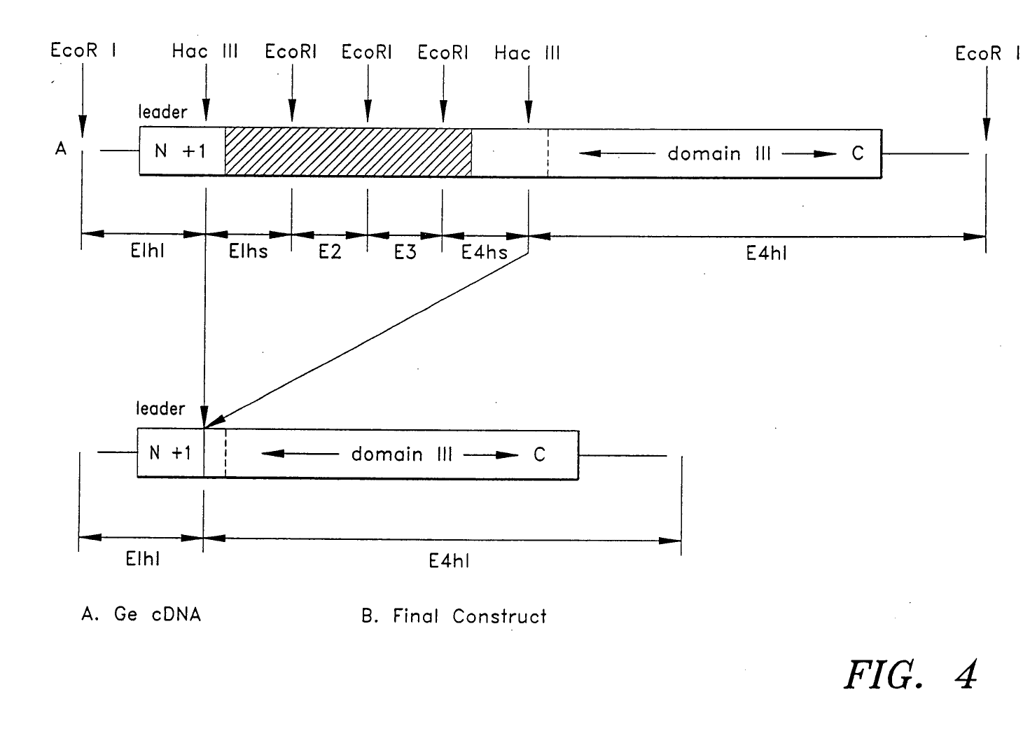

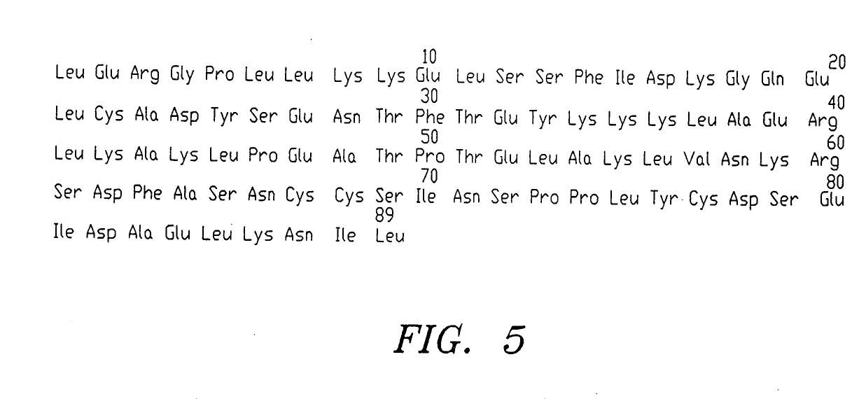

Since the glycosylation of a peptide requires a leader signal sequence for transfer of the peptide into the endoplasmic reticulum, the DNA segment of E1 containing the initiation codon (−16 Met) through the leader sequence to the +1 amino acid (Leu) of the native Gc protein should be introduced into the vector. Because this segment carries the initiation codon for the Gc protein, non-fusion vector, pVL1393 (Invitrogen, San Diego, Calif.) was used. A segment containing the initiation codon-leader sequence of the cDNA clone E1 and a segment coding for 85 C-terminal amino acids (the entire domain III plus 3′ non-coding stretch) of the cDNA clone E4 were ligated together and cloned into the EcoRI site of the insect virus pVL vector. To achieve this construct, both E1 and E4 were fragmented with Hae111 to yield two fragments each; E1h1 (87 bp), E1hs (33 bp) and E4hs (298 bp), E4h1 (450 bp), respectively. Both the larger fragments E1h1 and E4h1 were isolated electrophoretically, mixed with EcoR1-cut pVL, and ligated with T4 ligase, as shown in FIG. 4. This construct in correct orientation should express the entire domain III, a total of 89 amino acids, including the 4 amino acids of E1h1, also referred to herein as Cd as shown in FIG. 5. To obtain the correct construction, competent E. coli HB101 cells are transformed with pVL vector and selected for transformants on LB/ampicillin plates. DNA was prepared for sequencing procedures to determine which colony contains the construct with proper reading orientation by first searching for the 3′ poly dA stretch. Those clones with 3′ poly dA (from the poly A tail of mRNA) were then sequenced from the 5′ end to confirm correct orientation of the E1h1 fragment. It was then determined that the vector contained the entire construct (domain III) in the correct orientation.

3) Isolation of Recombinant Baculovirus, Purification of the Cloned Domain Peptide (Cd) and Enzymatic Generation of the Cloned Macrophage Activating Factor (CdMAF)

Monolayers (2.5×10⁶ cells in each of 25-cm² flasks) of Spodoptera frugiperda (Sf9) cells were co-transfected with cloned plasmid DNA (2 μg) and wild-type (AcMNPV) baculoviral DNA (10 μg) in 950 μl transfection buffer. Recombinant baculovirus plaques were isolated and used for production of the Gc domain III peptide in insect cells. This cloned domain with a molecular weight (MW) of 10,000 and 89 amino acids as shown in FIG. 5, was purified electrophoretically. Two μg of the cloned domain (Cd) peptide was treated with immobilized β-galactosidase and sialidase to yield a cloned macrophage activating factor, designated as CdMAF₁.

II. Cloning Procedure II: Fusion Vector

1) Cloning the Domain Responsible for Macrophage Activation (CdMAF)

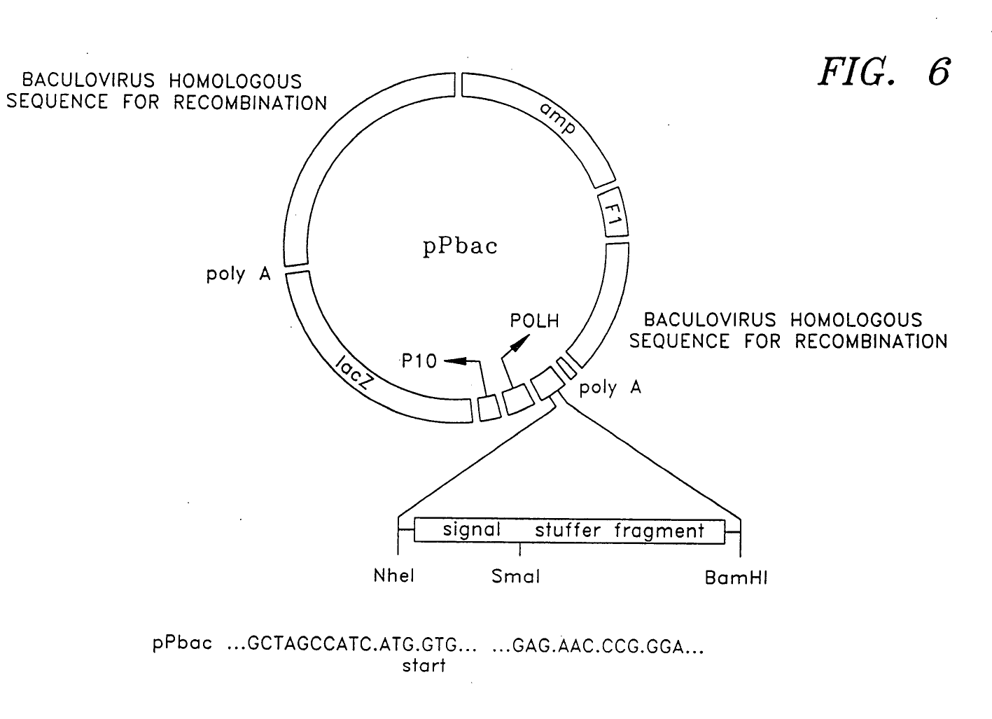

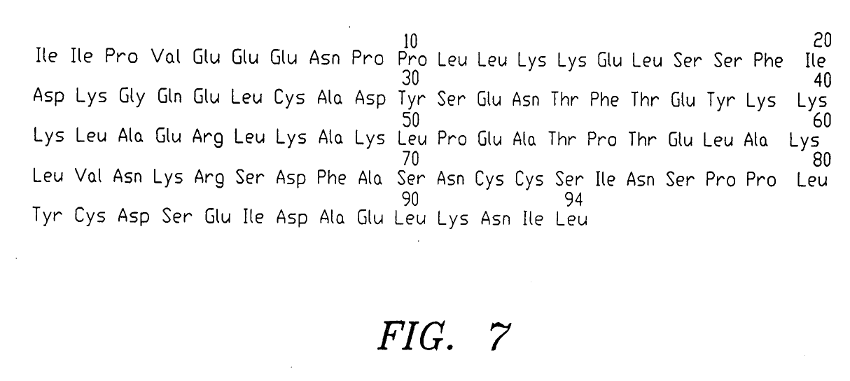

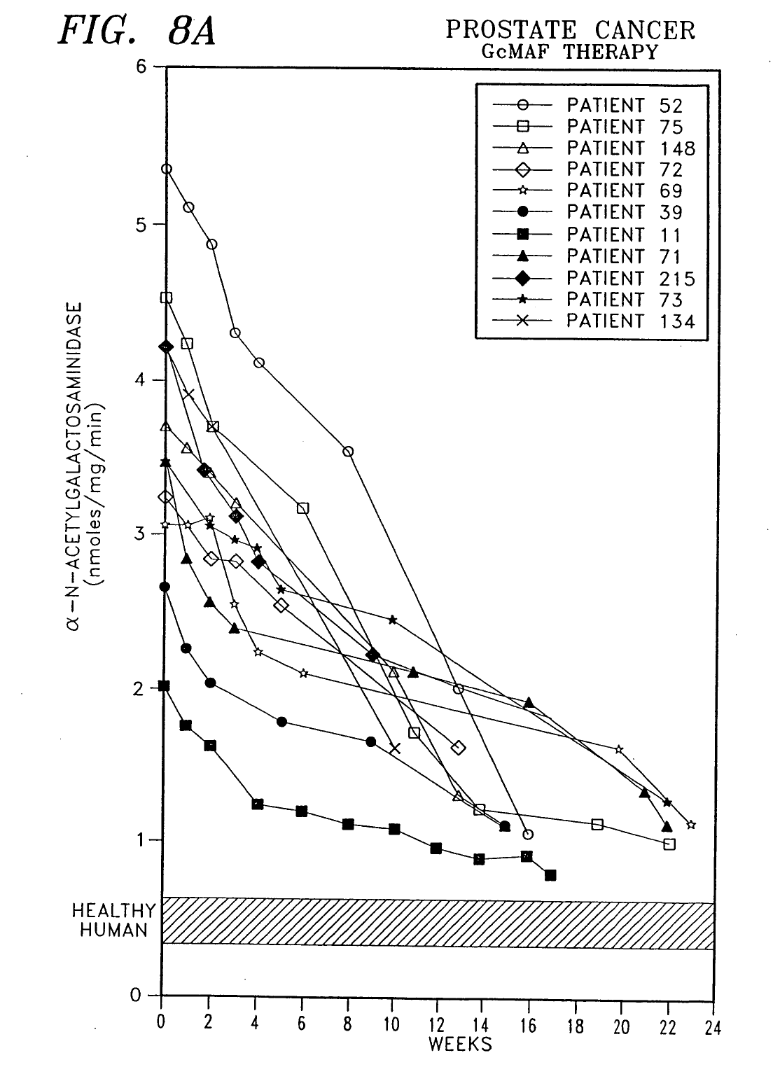

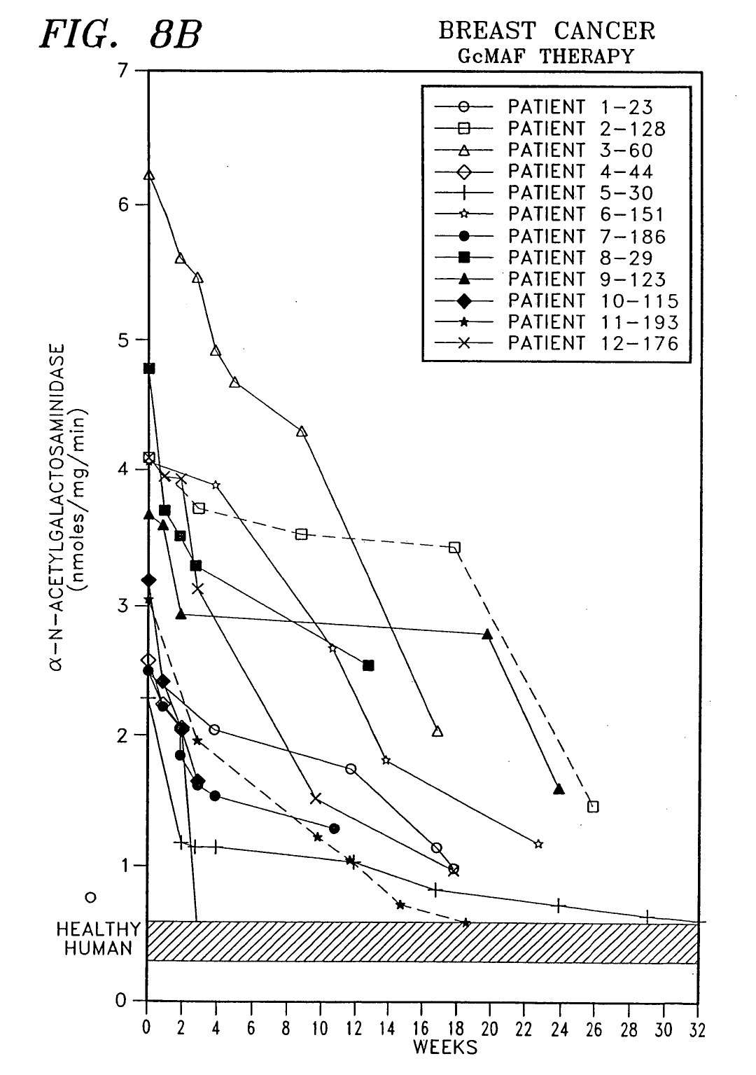

A baculovirus fusion vector, pPbac vector (Stratagene, La Jolla, Calif.), contains human placental alkaline phosphatase secretory signal sequences that direct the nascent cloned peptide chain toward the secretory pathway of the cells leading to secretion into culture media. The signal sequence is cleaved off by signal-sequence peptidase as the nascent cloned peptide is channeled toward the secretory pathway of the host insect cells leading to secretion of the cloned domain (Cd) peptide. FIG. 6 depicts that the vector carries the stuffer fragment for gene substitution and LacZ gene for identification of the gene insertion.

The stuffer fragment of pPbac vector was excised by digesting the vector DNA with restriction enzymes Sma1 and BamH1 and was removed by electroelution. The E4 cDNA fragment of the Gc protein was digested with Hae111 and BanH1, yielding a fragment practically the same as E4h1 (see FIG. 4). This fragment was mixed with the above ppbac vector and ligated with T4 ligase. This strategy not only fixes the orientation of ligation but also fuses the fragment with the reading frame. The E. coli DH5aF′ cells were transformed with the reaction mixture.

The cloned DNA insert was isolated from a number of colonies after digestion with Hae111 and BamH1. The insert was confirmed by sequencing. The sequence confirmed the correct orientation.

2) Isolation of Recombinant Baculovirus by Transfection of Sf9 Insect Cells with Wild Type Baculovirus and the Cloned DNA Insert

For transfection of insect cells (Spodoptera frugiperda, Sf9), cloned plasmid DNA, linear wild type (AcMNPV) baculoviral DNA and insectin liposomes (Invitrogen, San Diego, Calif.) have been used. Liposome-mediated transfection of insect cells is the most efficient transfection method available. For transfection to a monolayer of Sf9 cells (2×10⁶) in a 60 mm dish a mixture of the following was gently added:
3 μg cloned plasmid DNA
10 μl linear wild type baculovirus (AcMNPV) DNA (0.1 μg/μl)
1 ml medium
29 μl insectin liposomes

The dishes were incubated at room temperature for 4 hours with slow rocking. After transfection, the 1 ml of medium was added and incubated at 27° C. in a humidified environment for 48 hours. The resultant transfection lysate was plaque assayed. Purification of recombinant virus, isolation of the cloned domain peptide (Cd) and enzymatic generation of the cloned macrophage activating factor designated CdMAF₂ were described in the Cloning Procedure I. This CdMAF is composed of 94 amino acid residues as shown in FIG. 7, including 9 amino acids from the fusion vector and is referred to herein as CdMAF₂. Although CdMAF₂ has five amino acids more than the CdMAF₁ peptide derived from the non-fusion vector, they exhibited the same biological activities.

### Supporting Observations

A. Effects of Cloned Macrophage Activating Factors, GcMAFc and CdMAF on Cultured Phagocytes (Macrophages and Osteoclasts)

The three hour treatment of human macrophages or osteoclasts with picogram (pq) quantities of the cloned macrophage activating factors, GcMAFc and CdMAF, resulted in a greatly enhanced superoxide generating capacity of the phagocytes as shown in Table 1. The levels of phagocyte activation are similar to those of macrophage activation by GcMAF (Yamamoto et al., AIDS Res. Human Ret. 11:1373, 1995).

TABLE 1
Activation of phagocytes by in vitro treatment of GcMAF and its cloned
derivatives.

| | | nmole of superoxide | | |
|---|---|---|---|---|
| | | produced/min/10⁶ phagocytes | | |
| | Conc. | Human | Mouse peritoneal | Human |
| | pg/ml | macrophages* | macrophages | osteoclasts |
| GcMAFc | 0 | 0.07 | 0.06 | 0.03 |
| | 10 | 3.20 | 3.46 | 2.56 |
| | 100 | 5.18 | 5.08 | 4.22 |
| CdMAF | 0 | 0.01 | 0.02 | 0.08 |
| | 10 | 2.96 | 2.87 | 2.43 |
| | 100 | 4.26 | 4.53 | 4.09 |

*Peripheral blood monocytes/macrophages of cancer patients. Similar results were also observed when those of HIV-infected patients were used.

B. Activation of Mouse Peritoneal Macrophages by Administration of Cloned Macrophage Activating Factors, GcMAFc and CdMAF

One day post-administration of a picogram quantity (10 and 100 pg/mouse) of GcMAFc or CdMAF to BALB/c mice, peritoneal macrophages were isolated and assayed for superoxide generating capacity. As shown in Table 2, the macrophages were efficiently activated. These results are similar to those of macrophage activation by GcMAF (Naraparaju and Yamamoto, Immunol. Lett. 43:143, 1994; Yamamoto et al., AIDS Res. Human Ret. 11:1373, 1995).

TABLE 2
Activation of mouse peritoneal macrophages by administration
of cloned GcMAF derivatives.

| | | nmole of superoxide |
|---|---|---|
| | Dosage | produced/min/10⁶ phagocytes |
| | pg/mouse | Mouse peritoneal macrophages |
| GcMAFc | 0 | 0.05 |
| | 10 | 3.18 |
| | 100 | 5.23 |
| CdMAF | 0 | 0.03 |
| | 10 | 2.54 |
| | 100 | 4.23 |

C. Therapeutic Effects of GcMAF, GcMAFc or CdMAF on Tumor Bearing Mice and Osteopetrotic Mice

1) Therapeutic effects of GcMAF, GcMAFc or CdMAF on Ehrlich Ascites Tumor Bearing Mice

When BALB/c mice were administered with GcMAF, GcMAFc or CdMAF (100 pg/mouse) and received 10⁶ Ehrlich ascites tumor cells/mouse, they survived for at least 5 weeks. All the control mice received only the ascites tumor and died in approximately 14 days. When mice were administered with an additional 100 pg GcMAF/mouse 4 days post-transplantation, the tumor cells were completely eliminated (Table 3).

When mice were transplanted with 10⁵ Ehrlich ascites tumor cells/mouse and treated twice with GcMAF, GcMAFc or CdMAF (100/pg mouse) at 4 days and 8 days post-transplantation, all treated mouse groups survived over 65 days while the untreated 8 mouse groups all died at approximately 13 days (Groups 4 through 9 of Table 3).

TABLE 3
Therapeutic Effects of GcMAF and Cloned Derivatives
on Mice Bearing Ehrlich Ascites Tumor.

| | No. of | | No. of mice survived/ |
|---|---|---|---|
| Group | mice | Post-transplantation treatment | period |
| Group 1 | 6 mice | untreated control | 6 mice/13 ± 3 days |
| | 10 mice | day 0 100 pg GcMAF/mouse | 10 mice/36 ± 7 days |
| Group 2 | 6 mice | untreated control | 6 mice/14 ± days |
| | 10 mice | day 0 100 pg GcMAFc/mouse | 10 mice/35 ± 6 days |
| Group 3 | 6 mice | untreated control | 6 mice/14 ± 5 days |
| | 10 mice | day 0 100 pg CDMAF/mouse | 10 mice/34 ± 3 days |
| Group 4 | 8 mice | untreated control | 8 mice/15 ± 5 days |
| | 12 mice | day 0 100 pg GcMAF/mouse | |
| | | day 4 100 pg GcMAF/mouse | 12 mice/>65 days |
| Group 5 | 8 mice | untreated control | 8 mice/14 ± 5 days |
| | 12 mice | day 0 100 pg GcMAFc/mouse | |
| | | day 4 100 pg GcMAFc/mouse | 12 mice/>65 days |
| Group 6 | 8 mice | untreated control | 8 mice/14 ± 5 days |
| | 12 mice | day 0 100 pg CdMAF/mouse | |
| | | day 4 100 pg CdMAF/mouse | 12 mice/>65 days |
| Group 7 | 8 mice | untreated control | 8 mice/14 ± 4 days |
| | 8 mice | day 4 100 pg GcMAF/mouse | |
| | | day 8 100 pg GcMAF/mouse | 8 mice/>65 days |
| Group 8 | 8 mice | untreated control mice | 8 mice/13 ± 3 days |
| | 8 mice | day 4 100 pg GcMAFc/mouse | |
| | | day 8 100 pg GcMAFc/mouse | 8 mice/>65 days |
| Group 9 | 8 mice | untreated control | 8 mice/13 ± 5 days |
| | 8 mice | day 4 100 pg CdMAF/mouse | |
| | | day 8 100 pg CdMAF/mouse | 8 mice/>65 day |

With respect to the results of Table 3, GcMAF, GcMAFc or CdMAF was administered intraperitoneally for Groups 1 through 6, and GcMAF, GcMAFc or CdMAF was administered intramuscularly (systemically) for Groups 7 through 9; mice in all groups received 10⁵ tumor cells/mouse.

3) Therapeutic Effects of GcMAF and Cloned GcMAF Derivatives (GcMAFc and CdMAF) on Osteopetrotic Mice

Administration of GcMAFc or CdMAF to new born litters of osteopetrotic op/op mouse was performed by the weekly injection of 100 picograms for four weeks beginning from a day after birth. Mice were sacrificed at 28 days. The tibiae were removed from the treated and untreated control mice, longitudinally bisected, and examined under a dissecting microscope to measure the size of the bone marrow cavity. The cavity size was expressed as a percentage of the distance between the epiphyseal plates of the tibia. The untreated mouse group formed bone marrow with 30% of the total length of tibia. The treated mouse group experienced a 20% increased bone marrow formation over that of the untreated mouse group. This increased bone marrow cavity formation is an indication of osteoclast activation and increased osteoclastic bone resorption.

D. Therapeutic Effects of GcMAF, GcMAFc and CdMAF on Human Cancer and Virus Infected Patients.

1. Cancer Patients: Therapeutic Effect of GcMAF on Prostate, Breast and Colon Cancer and Adult Leukemia Patients

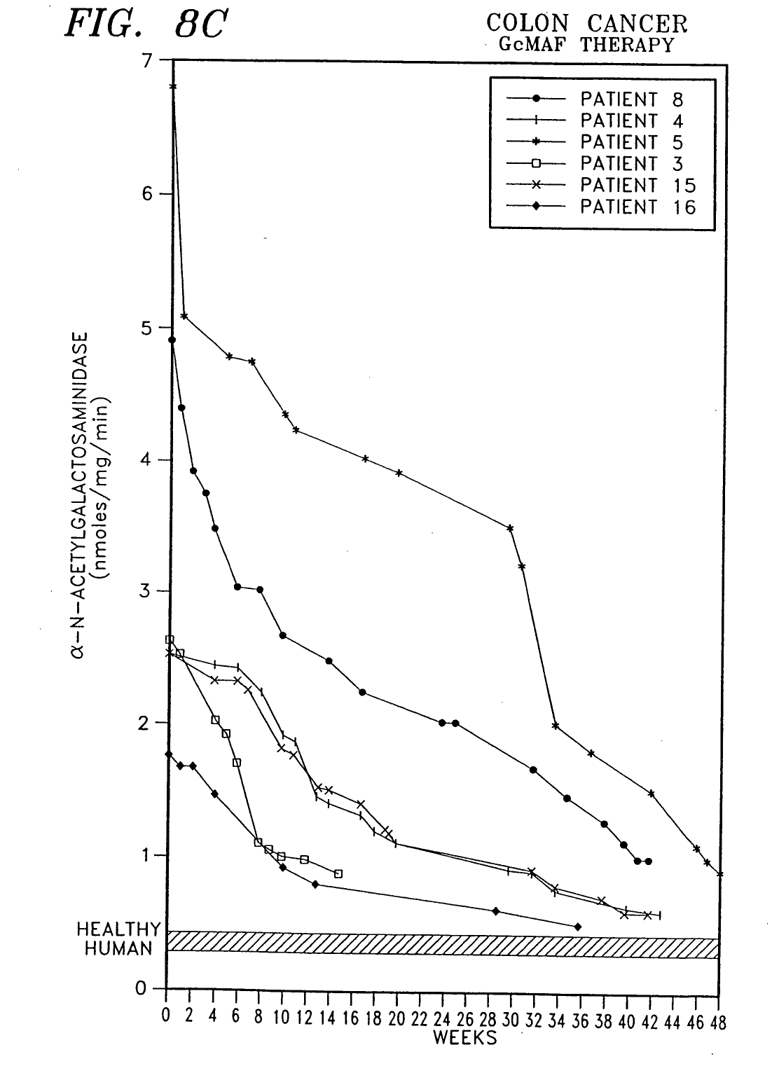

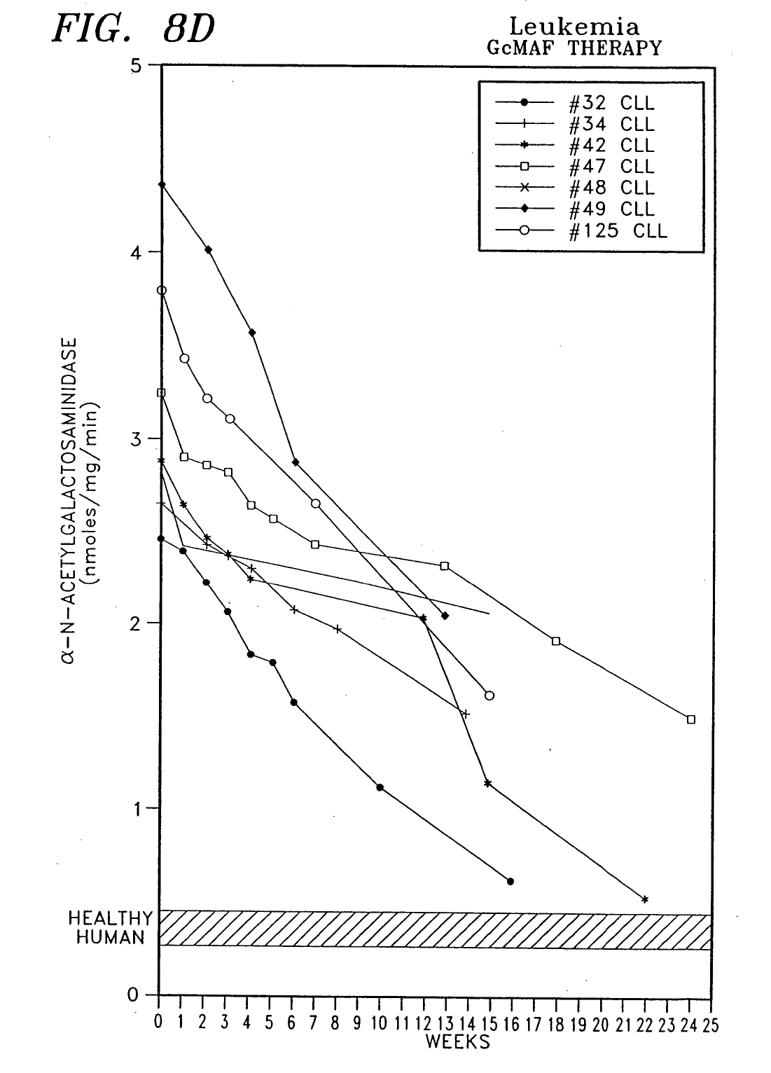

The administration of GcMAF (100 and 500 ng/human) to healthy volunteers resulted in the greatly enhanced activation of macrophages as measured by the 7-fold enhanced phagocytic capacity and the 15-fold superoxide generating capacity of macrophages. The administration of GcMAF showed no signs of any side effects to the recipients. Administration of various doses (100 pg to 10 ng/mouse) to a number of mice produced neither ill effects nor histological changes in various organs including liver, lung, kidney, spleen, brain, etc. When patients with various types of cancer were treated with GcMAF (100 ng/week), remarkable curative effects on various types of cancer were observed. The therapeutic efficacy of GcMAF on patients bearing various types of cancers was accessed by tumor specific serum α-N-acetylgalactosaminidase activity because the serum enzyme level is proportional to the total amount of cancerous cells (tumor burden). Curative effects of GcMAF on prostate, breast and colon cancer and leukemia are illustrated in FIGS. 8A to 8D. After 25 weekly administrations of 100 ng GcMAF the majority (>90%) of prostate and breast cancer patients exhibited insignificantly low levels of the serum enzyme. A similar result was also observed after 35 GcMAF administrations to colon cancer patients. Similar curative effects of GcMAF on lung, liver, stomach, brain, bladder, kidney, uterus, ovarian, larynx, esophagus, oral and skin cancers are observed. Thus, GcMAF appeared to be effective on a variety of cancers indiscriminately. However, GcMAF showed no evidence of side effects in patients after more than 6 months of therapy. This was also confirmed by blood cell counts profile, liver and kidney functions, etc.

When GcMAFc (100 ng/week) and CdMAF (100 ng/week) were administered to two prostate cancer patients each, curative effects similar to those of GcMAF were observed.

2. Virus Infected Patients

Treatment of peripheral blood macrophages of HIV-infected/AIDS patients with 100 pg GcMAF/ml resulted in a greatly enhanced macrophage activation (Yamamoto et al., AIDS Res. Human Ret. 11:1373, 1995). HIV-infected patients carry anti-HIV antibodies. HIV-infected cells express the viral antigens on the cell surface. Thus, macrophages have a potential to eliminate the infected cells via Fc-receptor mediated cell-killing/ingestion when activated.

Similarly, treatment of peripheral blood macrophages of patients chronically infected with Epstein-Barr virus (EBV) and with herpes zoster with 100 ng GcMAF/ml resulted in a greatly enhanced macrophage activation. Like HIV, EBV infects lymphocytes (B cells). Since these enveloped viruses code for α-N-acetylgalactosaminidase and infected cells secrete it into blood system. Thus this enzyme activity in patient sera can be used a s a prognostic index during therapy. After approximately 25 administrations of GcMAF (100 ng/week) to patients chronically infected with EBV and with herpes zoster, the enzyme activity decreased to that of healthy control levels. When GcMAFc (100 ng/week) or CdMAF (100 ng/week) was administered to EBV-infected patients, curative effects similar to those of GcMAF were observed.

E. Adjuvant Activities of GcMAF, GcMAFc and CdMAF for Immunization and Vaccinations

1. Rapid Increase of the Number of Antibody Secreting Cells (PFC) in Mice after Administration of GcMAF and Sheep Erythrocytes.

BALB/c mice were inoculated with SRBC 6 hours after the intraperitoneal administration of 50 pg GcMAF/mouse. At various intervals (1-5 days) after immunization, IgM-antibody secreting cells in the spleen were determined using the Jerne plaque assay (Jerne et al., Cell-bound antibodies, Wistar Institute Press, 1963). One day post-administration of GcMAF and SRBC produced 1.35×10⁴ PFC/spleen. Two days after administration of GcMAF and SRBC, the number of antibody secreting cells had increased to 8.2×10⁴ PFC/spleen. By the 4^{th} day the number of antibody secreting cells reached the maximal level (about 23.6×10⁴ PFC/spleen), as shown in Table 4. In contrast, mice that received an injection of SRBC alone produced about 3.8×10⁴ PFC/spleen, 4 days after SRBC-injection.

To ascertain the dose response, mice were injected with SRBC 6 hours after administration of various doses of GcMAF ranging from 1 to 50 pg/mouse. On the 4^{th} day post-administration of GcMAF and SRBC, the number of antibody secreting cells per spleen was determined by the Jerne plaque assay. On the 4^{th} day post-administration there was a commensurate increase in the number of plaque forming cells as the concentration of GcMAF was increased above 1 pg per mouse. At a GcMAF dose of 5, 10 and 50 pg/mouse, I detected 12.6×10⁴, 20.2×10⁴ and 24.3×10⁴ PFC/spleen, respectively.

TABLE 4
Time course studies on development of cells secreting antibody against
sheep erythrocytes (SRBC) in BALB/c mice after administration of
GcMAF and SRBC^{a}.

| | |
|---|---|
| After SRBC | Antibody secreting |
| immunization | cells/spleen (×10⁴) |

| (days) | SRBC only | GcMAF + SRBC |
|---|---|---|
| 1 | 0.01 ± 0.002 | 1.35 ± 0.21 |
| 2 | 0.08 ± 0.02 | 8.28 ± 1.26 |
| 3 | 1.18 ± 0.42 | 14.42 ± 2.32 |
| 4 | 3.86 ± 0.95 | 23.68 ± 6.05 |
| 5 | 2.15 ± 0.63 | 18.63 ± 3.43 |

^{a}Mice were inoculated with SRBC (10⁸ cells) 6 hr after administration of GcMAF (50 pg/mouse). The number of plaques (IgM secreting cells) was quantified microscopically on various days post-SRBC injection. The number of plaque-forming cells (PFC) per spleen is expressed as the mean value of triplicate assays ± SEM.

The invention will be illustrated in more detail with reference to the following Examples, but it should be understood that the present invention is not deemed to be limited thereto.

While the invention has been described in detail and with reference to specific examples thereof, it will be apparent to one skilled in the art that various changes and modifications can be made therein without departing from the spirit and scope thereof.

### References Cited

The following references are cited and their entire text is incorporated fully herein as are all references set forth in the specification.

### U.S. Patent Documents

U.S. Pat. Nos. 5,177,001 5,177,002 and 5,326,749 (Yamamoto).

### Other Publications

1. Jerne, N. K., Nordic, A. A. and Henry, C., The agar plaque technique for recognizing antibody producing cells. In Amos and Koprowski (eds). Cell-bound Antibody. Wistar Institute Press, Philadelphia, Pa. (1963).
2. Sato, M., Tanaka, H., Yamada, T. and Yamamoto, N., Persistent infection of BHK/WI-2 cells with rubella virus and characterization of rubella variants. Arch. Virology 54:333-343 (1977).
3. Reeves, J. D., August, C. S. Humbert, J. R. Weston, W. L., Host defense in infantile osteopetrosis, Pediatrics, 64:202 (1979)
4. Marks, S. C., Jr., Congenital osteopetrotic mutations as probes of the origin, s structure and function of osteoclasts, Clin, Orthop. 189:239-236 (1984).
5. Carbonell, L. F., Klowden, M. J. and Miller, L. K., Baculovirus-mediated expression of bacterial genes in dipteran and mammalian cells. J. Virol. 56:153-160 (1985).
6. Ngwenya, B. Z., and Yamamoto, N. Activation of peritoneal macrophages by lysophosphatidylcholine. Biochem. Biophys. Acta 839:9-15 (1985)
7. Cooke, N. E. and Haddad, J. G., Vitamin D binding protein (Gc-globulin). Endocrine Rev. 10:294-307 (1989).
8. Marks, S. C., Jr., Osteoclast biology: Lessons from mammalian mutations. Am. J. Med. Genet. 34:43-54 (1989).
9. Ngwenya, B. Z. and Yamamoto, N., Contribution of lysophosphatidylcholine treated nonadherent cells to mechanism of macrophage stimulation. Proc. Soc. Exp. Biol. Med. 193:118-124 (1990).
10. Ausubel, F. A., Brent, R., Kingston, R. E., Moore, D. D., Seidman, J. G., Smith, J. A. and Struhl, K. (eds), Expression of proteins in insert cells using baculoviral vectors. Current Protocols in Molecular Biology. Sections 16.8.1-16.11.7. Greene Publishing and Wiley-Interscience, New York (1990).
11. Yagi, F., Eckhardt, A. E. and Goldstein I. J., Glycosidases of Ehrlich ascites tumor cells and ascitic fluid-purification and substrate specificity of —N-acetylgalactosaminidase and -galactosidase. Arch. Biochem. Biophys. 280:61-67 (1990).
12. Yamamoto, N. and Homma, S., Vitamin D₃ binding protein (group-specific component, Gc) is a precursor for the macrophage activating signal from lysophosphatidylcholine-treated lymphocytes. Proc. Natl. Acad. Sci. USA 88:8539-8543 (1991).
13. Cooke, N. E. and David, E. V., Serum vitamin D-binding protein is a third member of the albumin and alpha-fetoprotein gene family. J. Clin. Invest. 76:2420-2424 (1985).
14. Haddad, J. G., Hu, Y. Z., Kowalski, M. A., Laramore, C., Ray, K., Robzyk, P. and Cooke, N. E., Identification of the sterol-and actin-binding domains of plasma vitamin D binding protein (Gc-globulin). Biochemistry 31:7174-7181 (1992).
15. Yamamoto, N. and Kumashiro, R., Conversion of vitamin D₃ binding protein (Group-specific component) to a macrophage activating factor by stepwise action of β-galactosidase of B cells and sialidase of T cells. J. Immunol. 151:27-94-2802 (1993).
16. Homma, S., Yamamoto, M. and Yamamoto, N., Vitamin D binding protein (group-specific component, Gc) is the sole serum protein required for macrophage activation after treatment of peritoneal cells with lysophosphatidylcholine. Immunol. Cell Biol. 71:249-257 (1993)
17. Yamamoto, N., Kumashiro, R., Yamamoto, M. Willett, N. P. and Lindsay, D. D., Regulation of inflammation-primed activation of macrophages by two serum factors, vitamin D₃-binding protein and albumin, Inf. 1 mm. 61:5388-5391 (1993).
18. Yamamoto, N., Lindsay, D. D., Naraparaju, V. R., Ireland, R. A. and Popoff, S. M., A defect in the inflammation-primed macrophage activation cascade in osteopetrotic (op) rats. J. Immunol. 152:5100-5107 (1994).
19. Yamamto, N., Willett, N. P. and Lindsay, D. D., Participation of serum proteins in the inflammation-primed activation of macrophages. Inflammation. 18:311-322 (1994).
20. Naraparaju, V. R. and Yamamoto, N., Roles of β-galactosidase of B lymphocytes and sialidase of T lymphocytes in inflammation-primed activation of macrophages. Immunol. Lett. 43:143-148 (1994).
21. Yamamoto, N., Naraparaju, V. R. and Srinivasula, S. M., Structural modification of serum vitamin D₃-binding protein and immunosuppression in HIV-infected patients. AIDS res. Human Ret. 11:1373-1378 (1995).
22. Yamamoto, N., Naraparaju, V. R. and Asbell, S. O. Deglycosylation of serum vitamin D₃-binding protein leads to immunosuppression in cancer patients. Cancer Research. 56:2827-2831 (1996).
23. Yamamoto, N., Naraparaju, V. R. and Urade, M. Prognostic utility of serum α-N-acetylgalactosaminidase and immunosuppression resulted from degylcosylation of serum Gc protein in oral cancer patients. Cancer Research. 57:295-299 (1997).
24. Koga, Y., Naraparaju, V. R. and Yamamoto, N. Antitumor effects of vitamin D₃-binding protein-derived macrophage activating factor on Ehrlich tumor bearing mice. Proc. Soc. Exp. Biol. Med. 220:20-26 (1999).

What is claimed is:

1. A process for producing a cloned macrophage activating factor (GcMAFc) comprising:
(a) cloning a Gc1 isoform into a baculovirus vector;
(b) expressing the cloned Gc1 isoform, thereby producing a cloned Gc1 protein, wherein the Gc1 protein comprises approximately 458 amino acids with a molecular weight of approximately 52,000 Da and 3 distinct domains;
(c) contacting the cloned Gc1 protein with immobilized beta galactosidase and sialidase; and
(d) obtaining the cloned macrophage activating factor (GcMAFc).

2. A process for producing a functional cloned macrophage activating factor (GcMAFc) comprising:
(a) cloning a Gc1 isoform into a baculovirus vector;
(b) expressing the Gc1 isoform, thereby producing a cloned Gc1 protein, wherein the cloned Gc1 protein comprises approximately 458 amino acids with a molecular weight of approximately 52,000 Da and 3 distinct domains;
(c) sequencing the cloned Gc1 peptide, thereby confirming that the cloned Gc1 protein is a cloned wild type Gc1 protein;
(d) contacting the cloned wild type Gc1 protein in vitro with immobilized beta galactosidase and sialidase, and
(e) obtaining the cloned macrophage activating factor (GcMAFc).
